(19) United States
(12) Patent Application Publication
(10) Pub. No.: US 2005/0191652 A1
(43) Pub. Date: Sep. 1, 2005

(54) Identification of genetic forms of a gene that leads to high risk for parkinson disease

(76) Inventors: Jeffery M. Vance, Chapel Hill, NC (US); Eden Martin, Hillsborough, NC (US); William Scott, Durham, NC (US); Margaret A. Pericak-Vance, Chapel Hill, NC (US); Mike Hauser, Durham, NC (US)

Correspondence Address: MYERS BIGEL SIBLEY & SAJOVEC, PO BOX 37428, RALEIGH, NC, 27627, US

(21) Appl. No.: 10/979,297

(22) Filed: Nov. 2, 2004

Related U.S. Application Data
(60) Provisional application No. 60/516,861, filed on Nov. 3, 2003.

(51) Int. Cl. C12Q 1/68

(52) U.S. Cl. 435/6

(57) ABSTRACT

The present invention discloses methods of screening a subject for Parkinson's disease comprising detecting the presence or absence of a marker or functional polymorphism associated with a gene linked to Parkinson's disease.

### CROSS REFERENCE TO RELATED APPLICATION

This application claims priority to U.S. Provisional Application Ser. No. 60/516,861, which was filed in the United States Patent and Trademark Office on Nov. 3, 2003, the disclosure of which is incorporated herein by reference in its entirety.

### FIELD OF THE INVENTION

This invention concerns methods of screening for Parkinson's disease by the screening of genetic risk factors. More particularly, this invention relations to single nucleotide polymorphism that are associated with an increased risk of getting Parkinson's disease.

### BACKGROUND OF THE INVENTION

Genetic studies of common complex neurodegenerative diseases, such as Alzheimer's disease and Parkinson's disease, have focused on the identification of risk genes as targets for development of new treatments and improved diagnoses. This approach has identified the amyloid precursor protein (APP) (Goate et al., Nature 349:704-706 (1991)), presenilin 1 (PS1) (Sherrington et al., Nature 375:754-760 (1995)), presenilin 2 (PS2) (Levy-Lahad et al., Science 269:973-977 (1995); Rogaev et al., Nature 376:775-778 (1995)), and apolipoprotein E (APOE) (Corder et al., Science 261:921-923 (1993)) genes as contributing to risk in Alzheimer's disease. Three genes have been identified to associate with risk in Parkinson's disease: α-synuclein (Polymeropoulos et al., Science 274:1197-1199 (1996)) for rare autosomal dominant early-onset Parkinson's disease, Parkin (Abbas et al., Hum Mol Genet 8:567-574 (1999)) for rare autosomal recessive juvenile parkinsonism and autosomal recessive early-onset Parkinson's disease, and tau (Martin et al., JAMA 286:2245-2250 (2001)) for classic Parkinson's disease. Genomic screens in both Parkinson's disease (Destefano et al., Neurology 57:1124-1126 (2001); Scott et al., JAAM 286:2239-2244 (2001)) and Alzheimer's disease (Kehoe et al., Hum Mol Genet 8:237-245 (1999); Pericak-Vance et al., Exp Gerontol 35:1343-1352 (2000) have recently localized additional but, as yet, unknown risk genes.

Parkinson's disease is a neurodegenerative, late-AAO disorders. Dementia is a major factor in Parkinson's disease. Parkinson's disease also exhibits degeneration of cholinergic neurons in the nucleus basalis of Meynert.

Identification of further genes would open new avenues of research with the potential to delay onset beyond the natural life span. Present knowledge about genes contributing to AAO in neurodegenerative diseases clearly lags behind the understanding of genes contributing to risk. Recently, there has been growing interest in using AAO information as a quantitative trait, to identify genes that influence onset of disease (Daw et al., Am J Hum Genet 64:839-851 (1999), Daw et al., Am J Hum Genet 66:196-204 (2000); Duggirala et al. Am J Hum Genet 64:1127-1140 (1999)). Rapid development of methods of mapping quantitative trait loci (QTLs) for general pedigrees (Goldgar, Am J Hum Genet 47:957-967 (1990); Amos, Am J Hum Genet 54:535-543 (1994); Blangero et al. Genet Epidemiol 14:959-964 (1997)) has now made the search for novel genes affecting AAO feasible. Thus, there is a continued need to develop new genetic linkages and markers as well as identifying new functional polymorphisms that are associated with Parkinson's disease.

### SUMMARY OF THE INVENTION

The present invention discloses methods of screening a subject for Parkinson's disease. The method comprises the steps of: detecting the presence or absence of a marker for Parkinson's disease, or a functional polymorphism associated with a gene linked to Parkinson's disease, with the presence of such a marker or functional polymorphism indicating that subject is afflicted with or at risk of developing Parkinson's disease. The detecting step may include detecting whether the subject is heterozygous or homozygous for the marker and/or functional polymorphism, with subjects who are at least heterozygous for the functional polymorphism being at increased risk for Parkinson's disease. The step of detecting the presence or absence of the marker or functional polymorphism may include the step of detecting the presence or absence of the marker or functional polymorphism in both chromosomes of the subject (i.e., detecting the presence or absence of one or two alleles containing the marker or functional polymorphism). More than one copy of a marker or functional polymorphism (i.e., subjects homozygous for the functional polymorphism) may indicate greater risk of Parkinson's disease as compared to heterozygous subjects.

A further aspect of the present invention is the use of a means of detecting a marker, functional polymorphism or mutation as described herein in screening a subject for Parkinson's disease as described herein.

The foregoing and other objects and aspects of the present invention are explained in detail in the drawings herein and the specification set forth below.

### BRIEF DESCRIPTION OF THE DRAWINGS

FIG. 1 depicts chromosome 8.

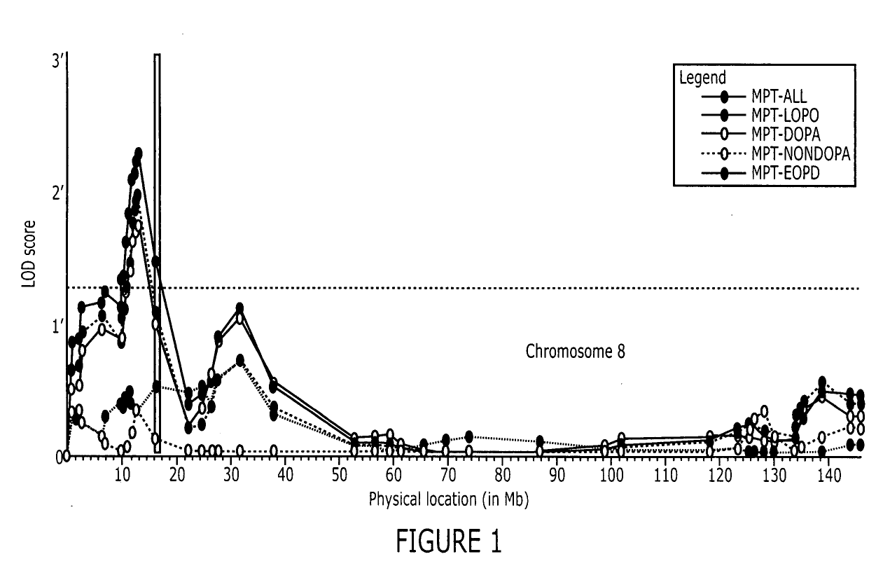

FIG. 2 illustrates the locations on FGF20 where the single nucleotide polymorphisms reside.

FIG. 3 demonstrates the alignment of human and mouse FGF20 3′UTR for rs1721100 and 8p0215.

### DETAILED DESCRIPTION OF THE EMBODIMENTS

As noted above, the present invention provides a method of screening (e.g., diagnosing, detecting, determining or prognosing) for Parkinson's disease in a subject. Subjects with which the present invention is concerned are primarily human subjects, including male and female subjects of any age or race.

The term “age at onset” (AAO) refers to the age at which a subject is affected with a particular disease.

The term “Parkinson's disease (PD) as used herein is intended to encompass all types of Parkinson's disease.

“Screening” as used herein refers to a procedure used to evaluate a subject for risk of Parkinson's disease. It is not required that the screening procedure be free of false positives or false negatives, as long as the screening procedure is useful and beneficial in determining which of those individuals within a group or population of individuals are at increased risk of Parkinson's disease. A screening procedure may be carried out for both prognostic and diagnostic purposes (i.e., prognostic methods and diagnostic methods).

“Prognostic method” refers to methods used to help predict, at least in part, the course of a disease. For example, a screening procedure may be carried out on a subject that has not previously been diagnosed with Parkinson's disease, or does not show substantial disease symptoms, when it is desired to obtain an indication of the future likelihood that the subject will be afflicted with Parkinson's disease. In addition, a prognostic method may be carried out on a subject previously diagnosed with Parkinson's disease when it is desired to gain greater insight into how the disease will progress for that particular subject (e.g., the likelihood that a particular patient will respond favorably to a particular drug treatment, or when it is desired to classify or separate Parkinson's disease patients into distinct and different subpopulations for the purpose of conducting a clinical trial thereon). A prognostic method may also be used to determine whether a person will respond to a particular drug.

“Diagnostic method” as used herein refers to screening procedures carried out on a subject that has previously been determined to be at risk for a particular neurodegenerative disorder due to the presentation of symptoms or the results of another (typically different) screening test.

“Functional polymorphism” as used herein refers to a change in the base pair sequence of a gene that produces a qualitative or quantitative change in the activity of the protein encoded by that gene (e.g., a change in specificity of activity; a change in level of activity). The presence of a functional polymorphism indicates that the subject is at greater risk of developing a particular disease as compared to the general population. For example, the patient carrying the functional polymorphism may be particularly susceptible to chronic exposure to environmental toxins that contribute to Parkinson's disease. The term “functional polymorphism” includes mutations, deletions and insertions.

A “present” functional polymorphism as used herein (e.g., one that is indicative of or a risk factor for Parkinson's disease) refers to the nucleic acid sequence corresponding to the functional polymorphism that is found less frequently in the general population relative to Parkinson's disease as compared to the alternate nucleic acid sequence or sequences found when such functional polymorphism is said to be “absent”.

“Mutation” as used herein sometimes refers to a functional polymorphism that occurs in less than one percent of the population, and is strongly correlated to the presence of a gene (i.e., the presence of such mutation indicating a high risk of the subject being afflicted with a disease). However, “mutation” is also used herein to refer to a specific site and type of functional polymorphism, without reference to the degree of risk that particular mutation poses to an individual for a particular disease.

“Linked” as used herein refers to a region of a chromosome that is shared more frequently in family members affected by a particular disease, than would be expected by chance, thereby indicating that the gene or genes within the linked chromosome region contain or are associated with a marker or functional polymorphism that is correlated to the presence of, or risk of, disease. Once linkage is established association studies (linkage disequilibrium) can be used to narrow the region of interest or to identify the risk conferring gene for Parkinson's disease.

“Associated with” when used to refer to a marker or functional polymorphism and a particular gene means that the functional polymorphism is either within the indicated gene, or in a different physically adjacent gene on that chromosome. In general, such a physically adjacent gene is on the same chromosome and within 2, 3, 5, 10 or 15 centimorgans of the named gene (i.e., within about 1 or 2 million base pairs of the named gene). The adjacent gene may span over 5, 10 or even 15 megabases.

A unit of measure of recombination frequency. One centimorgan is equal to a 1% chance that a marker at one genetic locus will be separated from a marker at a second locus due to crossing over in a single generation. In humans, 1 centimorgan is equivalent, on average, to one million base pairs.

Markers (e.g., genetic markers such as restriction fragment length polymorphisms and simple sequence length polymorphisms) may be detected directly or indirectly. A marker may, for example, be detected indirectly by detecting or screening for another marker that is tightly linked (e.g., is located within 2 or 3 centimorgans) of that marker. Additionally, the adjacent gene may be found within an approximately 15 cM linkage region surrounding the chromosome thus spanning over 5, 10 or even 15 megabases.

The presence of a marker or functional polymorphism associated with a gene linked to Parkinson's disease indicates that the subject is afflicted with Parkinson's disease or is at risk of developing Parkinson's disease. A subject who is “at increased risk of developing Parkinson's disease” is one who is predisposed to the disease, has genetic susceptibility for the disease or is more likely to develop the disease than subjects in which the detected functional polymorphism is absent. While the methods described herein may be employed to screen for any type of idiopathic Parkinson's disease, a primary application is in screening for late-onset or early-onset Parkinson's disease.

The marker or functional polymorphism may also indicate “age of onset” of Parkinson's disease, particularly subjects at risk for Parkinson's disease, with the presence of the marker indicating an earlier age of onset for Parkinson's disease.

Suitable subjects include those who have not previously been diagnosed as afflicted with Parkinson's disease, those who have previously been determined to be at risk of developing Parkinson's disease, and those who have been initially diagnosed as being afflicted with Parkinson's disease where confirming information is desired. Thus, it is contemplated that the methods described herein be used in conjunction with other clinical diagnostic information known or described in the art which are used in evaluation of subjects with Parkinson's disease or suspected to be at risk for developing such disease.

The detecting step may be carried out in accordance with known techniques (See, e.g., U.S. Pat. Nos. 6,027,896 and 5,508,167 to Roses et al.), such as by collecting a biological sample containing DNA from the subject, and then determining the presence or absence of DNA encoding or indicative of the functional polymorphism in the biological sample (e.g., the Parkin gene exon 3 deletion mutation described herein). Any biological sample which contains the DNA of that subject may be employed, including tissue samples and blood samples, with blood cells being a particularly convenient source.

Determining the presence or absence of DNA encoding a particular functional polymorphism may be carried out with an oligonucleotide probe labeled with a suitable detectable group, and/or by means of an amplification reaction such as a polymerase chain reaction or ligase chain reaction (the product of which amplification reaction may then be detected with a labeled oligonucleotide probe or a number of other techniques). Further, the detecting step may include the step of detecting whether the subject is heterozygous or homozygous for the particular functional polymorphism. Numerous different oligonucleotide probe assay formats are known which may be employed to carry out the present invention. See, e.g., U.S. Pat. No. 4,302,204 to Wahl et al.; U.S. Pat. No. 4,358,535 to Falkow et al.; U.S. Pat. No. 4,563,419 to Ranki et al.; and U.S. Pat. No. 4,994,373 to Stavrianopoulos et al. (applicants specifically intend that the disclosures of all U.S. Patent references cited herein be incorporated herein by reference). The oligonucleotides may be used to hybridize to the nucleic acids disclosed in the present application. The oligonucleotides may be from 2 to 100 nucleotides and preferably from 5 to 50 bases.

Amplification of a selected, or target, nucleic acid sequence may be carried out by any suitable means. See generally, Kwoh et al., Am. Biotechnol. Lab. 8, 14-25 (1990). Examples of suitable amplification techniques include, but are not limited to, polymerase chain reaction, ligase chain reaction, strand displacement amplification (see generally G. Walker et al., Proc. Natl. Acad. Sci. USA 89, 392-396 (1992); G. Walker et al., Nucleic Acids Res. 20, 1691-1696 (1992)), transcription-based amplification (see D. Kwoh et al., Proc. Natl. Acad. Sci. USA 86, 1173-1177 (1989)), self-sustained sequence replication (or “3SR”) (see J. Guatelli et al., Proc. Natl. Acad. Sci. USA 87, 1874-1878 (1990)), the Qβ replicase system (see P. Lizardi et al., BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra). Polymerase chain reaction is currently preferred.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques, or by direct visualization on a gel. When PCR conditions allow for amplification of all allelic types, the types can be distinguished by hybridization with an allelic specific probe, by restriction endonuclease digestion, by electrophoresis on denaturing gradient gels, or other techniques.

DNA amplification techniques such as the foregoing can involve the use of a probe, a pair of probes, or two pairs of probes which specifically bind to DNA containing the functional polymorphism, but do not bind to DNA that does not contain the functional polymorphism. Alternatively, the probe or pair of probes could bind to DNA that both does and does not contain the functional polymorphism, but produce or amplify a product (e.g., an elongation product) in which a detectable difference may be ascertained (e.g., a shorter product, where the functional polymorphism is a deletion mutation). Such probes can be generated in accordance with standard techniques from the known sequences of DNA in or associated with a gene linked to Parkinson's disease or from sequences which can be generated from such genes in accordance with standard techniques.

It will be appreciated that the detecting steps described herein may be carried out directly or indirectly. Other means of indirectly determining allelic type include measuring polymorphic markers that are linked to the particular functional polymorphism, as has been demonstrated for the VNTR (variable number tandem repeats) and the ApoB alleles (Decorter et al., DNA & Cell Biology 9(6), 461-69 (1990), and collecting and determining differences in the protein encoded by a gene containing a functional variant, as described for ApoE4 in U.S. Pat. Nos. 5,508,167 and 6,027,896 to Roses et al.

One form of genetic analysis is analysis centered on elucidation of single nucleic acid polymorphisms or (“SNPs”). Factors favoring the usage of SNPs are their high abundance in the human genome (especially compared to short tandem repeats, (STRs)), their frequent location within coding or regulatory regions of genes (which can affect protein structure or expression levels), and their stability when passed from one generation to the next (Landegren et al., Genome Research, Vol. 8, pp. 769-776, 1998).

A SNP is defined as any position in the genome that exists in two variants and the most common variant occurs less than 99% of the time. In order to use SNPs as widespread genetic markers, it is crucial to be able to genotype them easily, quickly, accurately, and cost-effectively. It is of great interest to type both large sets of SNPs in order to investigate complex disorders where many loci factor into one disease (Risch and Merikangas, Science, Vol. 273, pp. 1516-1517, 1996), as well as small subsets of SNPs previously demonstrated to be associated with known afflictions.

Numerous techniques are currently available for typing SNPs (for review, see Landegren et al., Genome Research, Vol. 8, pp. 769-776, 1998), all of which require target amplification. They include direct sequencing (Carothers et al., BioTechniques, Vol. 7, pp. 494-499, 1989), single-strand conformation polymorphism (Orita et al., Proc. Natl. Acad. Sci. USA, Vol. 86, pp. 2766-2770, 1989), allele-specific amplification (Newton et al., Nucleic Acids Research, Vol. 17, pp. 2503-2516, 1989), restriction digestion (Day and Humphries, Analytical Biochemistry, Vol. 222, pp. 389-395, 1994), and hybridization assays. In their most basic form, hybridization assays function by discriminating short oligonucleotide reporters against matched and mismatched targets. Due to difficulty in determining optimal denaturation conditions, many adaptations to the basic protocol have been developed. These include ligation chain reaction (Wu and Wallace, Gene, Vol. 76, pp. 245-254, 1989) and minisequencing (Syvanen et al., Genomics, Vol. 8, pp. 684-692, 1990). Other enhancements include the use of the 5′-nuclease activity of Taq DNA polymerase (Holland et al., Proc. Natl. Acad. Sci. USA, Vol. 88, pp. 7276-7280, 1991), molecular beacons (Tyagi and Kramer, Nature Biotechnology, Vol. 14, pp. 303-308, 1996), heat denaturation curves (Howell et al., Nature Biotechnology, Vol. 17, pp. 87-88, 1999) and DNA “chips” (Wang et al., Science, Vol. 280, pp. 1077-1082, 1998). While each of these assays are functional, they are limited in their practical application in a clinical setting.

An additional phenomenon discovered to be useful in distinguishing SNPs is the nucleic acid interaction energies or base-stacking energies derived from the hybridization of multiple target specific probes to a single target. (see R. Ornstein et al., “An Optimized Potential Function for the Calculation of Nucleic Acid Interaction Energies”, Biopolymers, Vol. 17, 2341-2360 (1978); J. Norberg and L. Nilsson, Biophysical Journal, Vol. 74, pp. 394 -402, (1998); and J. Pieters et al., Nucleic Acids Research, Vol. 17, no. 12, pp. 4551-4565 (1989)). This base-stacking phenomenon is used in a unique format in the current invention to provide highly sensitive Tm differentials allowing the direct detection of SNPs in a nucleic acid sample.

Additional methods have been used to distinguish nucleic acid sequences in related organisms or to sequence DNA. For example, U.S. Pat. No. 5,030,557 by Hogan et al. disclosed that the secondary and tertiary structure of a single stranded target nucleic acid may be affected by binding “helper” oligonucleotides in addition to “probe” oligonucleotides causing a higher Tm to be exhibited between the probe and target nucleic acid. That application however was limited in its approach to using hybridization energies only for altering the secondary and tertiary structure of self-annealing RNA strands which if left unaltered would tend to prevent the probe from hybridizing to the target.

With regard to DNA sequencing, K. Khrapko et al., Federation of European Biochemical Societies Letters, Vol. 256, no. 1,2, pp. 118-122 (1989), for example, disclosed that continuous stacking hybridization resulted in duplex stabilization. Additionally, J. Kieleczawa et al., Science, Vol. 258, pp. 1787-1791 (1992), disclosed the use of contiguous strings of hexamers to prime DNA synthesis wherein the contiguous strings appeared to stabilize priming. Likewise, L. Kotler et al., Proc. Natl. Acad. Sci. USA, Vol. 90, pp. 4241-4245, (1993) disclosed sequence specificity in the priming of DNA sequencing reactions by use of hexamer and pentamer oligonucleotide modules. Further, S. Parinov et al., Nucleic Acids Research, Vol. 24, no. 15, pp. 2998-3004, (1996), disclosed the use of base-stacking oligomers for DNA sequencing in association with passive DNA sequencing microchips. Moreover, G. Yershov et al., Proc. Natl. Acad. Sci. USA, Vol. 93, pp. 4913-4918 (1996), disclosed the application of base-stacking energies in SBH on a passive microchip. In Yershov's example, 10-mer DNA probes were anchored to the surface of the microchip and hybridized to target sequences in conjunction with additional short probes, the combination of which appeared to stabilize binding of the probes. In that format, short segments of nucleic acid sequence could be elucidated for DNA sequencing. Yershov further noted that in their system the destabilizing effect of mismatches was increased using shorter probes (e.g., 5-mers). Use of such short probes in DNA sequencing provided the ability to discern the presence of mismatches along the sequence being probed rather than just a single mismatch at one specified location of the probe/target hybridization complex. Use of longer probes (e.g., 8-mer, 10-mer, and 13-mer oligos) were less functional for such purposes.

An additional example of methodologies that have used base-stacking in the analysis of nucleic acids includes U.S. Pat. No. 5,770,365 by Lane et al., wherein is disclosed a method of capturing nucleic acid targets using a unimolecular capture probe having a single stranded loop and a double stranded region which acts in conjunction with a binding target to stabilize duplex formation by stacking energies.

Kits useful for carrying out the methods of the present invention will, in general, comprise one or more oligonucleotide probes and other reagents for carrying out the methods as described above, such as restriction enzymes, optionally packaged with suitable instructions for carrying out the methods. Additionally, they may include methods for detecting for the presence or absence of at least one functional polymorphism as described herein and instructions for observing that the subject is or was afflicted with or is or was at increased risk of developing Parkinson's disease if at least one of the functional polymorphisms is detected.

The present invention also provides a method of conducting a clinical trial on a plurality of human subjects or patients. Such methods advantageously permit the refinement of the patient population so that advantages of particular treatment regimens (typically administration of pharmaceutically active organic compound active agents) can be more accurately detected, particularly with respect to particular sub-populations of patients. In general, such methods comprise administering a test active agent or therapy to a plurality of subjects (a control or placebo therapy typically being administered to a separate but similarly characterized plurality of subjects) and detecting the presence or absence of at least one mutation or polymorphism as described above in the plurality of subjects. The polymorphisms may be detected before, after, or concurrently with the step of administering the test therapy. The influence of one or more detected polymorphisms or absent polymorphisms on the test therapy can then be determined on any suitable parameter or potential treatment outcome or consequence, including but not limited to: the efficacy of said therapy, lack of side effects of the therapy, etc.

The pathogenic process responsible for the loss of dopaminergic neurons within the substantia nigra of Parkinson's disease patients is not well understood. However, there is strong evidence to support the involvement of fibroblast growth factor 20 (FGF20) in the survival of dopaminergic neurons. FGF20 belongs to a highly conserved family of growth factor polypeptides that regulate CNS development and function. Additionally, FGF20 is involved in differentiation of rat stem cells into dopaminergic cells. FGF20 is preferentially expressed in rat substantia nigra tissue. The human homologue has been mapped to 8p21.3 to 8p22.

The present invention is explained in greater detail in the Examples that follow. These examples are intended as illustrative of the invention and are not to be taken as limiting thereof.

### EXAMPLES

Single nucleotide polymorphisms found in the public record (rs 1989754, rs1989756, and rs1721100) were tested. It was found that the SNP rs1989754 was significantly associated with increasing risk for Parkinson Disease (PD) (See Table 1).

TABLE 1
Results of single locus and genotype association analyses

| | PDTsum | genoPDT |
|---|---|---|
| Overall | | |
| 8P0217 | 0.1616 | 0.4077 |
| rs1989756 | 0.3942 | 0.4355 |
| rs1989754 | 0.0006 | 0.0056 |
| rs1721100 | 0.0196 | 0.0713 |
| 8p0215 | 0.0008 | 0.0004 |
| Hx+ | | |
| 8P0217 | 0.2902 | 0.5984 |
| rs1989756 | 0.1218 | 0.2111 |
| rs1989754 | 0.0033 | 0.0249 |
| rs1721100 | 0.2058 | 0.3344 |
| 8p0215 | 0.0047 | 0.0042 |

Additionally, using DNA sequencing analysis of control DNA, a new polymorphism was discovered called 8p0215. Association testing demonstrated that this SNP is also highly associated with an increased risk with getting Parkinson disease (See Table 1). The “2” allele, which corresponds to the T allele, is the allele associated with increased risk for Parkinson disease. Another SNP, 8p0217, was discovered using the same technique.

The location for 8p215 in the FGF20 cDNA sequence lies at position 817C>T in the cDNA. The location is shown below. The first base is the MET codon should be numbered 1+.

[image]

[image]

[image]

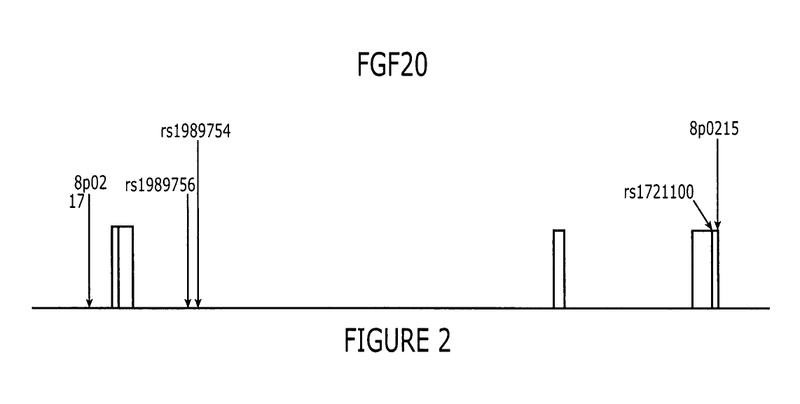

It was determined that SNP rs1989754 lies in the first intron, and 8p0215 lies in the 3′ UTR of FGF20. (FIG. 2). This SNP is in an intronic area, thus it is best noted by the rs designation. The actual sequence number may change with each number thus one skilled in the art will appreciate that the number may change. The sequence shown below is shown flanking the polymorphism as is characterized as dbSNP rs# rs1989754, has the genomic location Chromosome 8:16,938,312, was characterized by the Sanger Center and was submitted on Oct. 13, 2003. The flanking sequence information and observed SNP are as follows:

5′flank: tcctttgaca ttgctagcag gttaactaat agaatggaaa cttcagctat ggggaaagat
cctgggatat tagaaccgga gagcacccca tctttgtaca gaaaactaag cctcagactg
atgaaggcac tttctagtta cacagctagt gaggaagtca ttaacaggag agaccctccc
gatctagtat cttaacagac actgccttaa caatcattct cttgtttctt ttaacccctt
ctcttcccag gcactgccgg aggtattctg aaacacgtcc gtctgtgttc ccacccatat
cttctttcgc tttcccattt cctctttcct aaagtcgata ccaagatact tgctttca

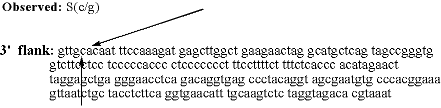

The rs1989754 SNP is located in a HIF1alpha binding site, which is a known enducer for expression during hypoxia. The letters in bold (CGTG) are the consensus binding site for HIF1alpha binding. Variation introduced by the rs1989754 SNP disrupts the binding site, with the allele causing an increase in risk with PD disrupting the site, and the allele associated with decreased risk, keeping the site as the consensus sequence.

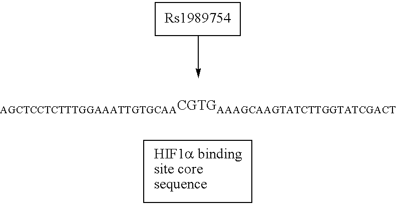

This implies that FGF20 could be induced to express during hypoxia. Using PC12 cells and hypoxic conditions, we demonstrated for the first time that FGF20 is indeed induced by hypoxia.

TABLE 2
Haplotype analysis of FGF20
Estimated haplotypes in the overall dataset

| | SNPs genotyped | | | | | | | | |
|---|---|---|---|---|---|---|---|---|---|
| | 8p0217 | rs1989756 | rs1989754 | rs1721100 | 8p0215 | #Families | Frequency | Z | p-value |
| h1 | 1 | 2 | 1 | 2 | 1 | 228 | 0.42 | −3.318 | 0.0009 |
| h2 | 2 | 2 | 2 | 2 | 1 | 205 | 0.21 | 0.294 | ns |
| h3 | 2 | 2 | 2 | 1 | 1 | 179 | 0.19 | 0.691 | ns |
| h4 | 1 | 2 | 2 | 1 | 2 | 80 | 0.08 | 3.587 | 0.0003 |
| h5 | 2 | 1 | 2 | 2 | 1 | 89 | 0.06 | 0.465 | ns |
| h6 | 1 | 2 | 2 | 2 | 1 | 11 | 0.008 | −0.488 | ns |
| h7 | 2 | 1 | 2 | 1 | 1 | 25 | 0.005 | −0.254 | ns |
| | | | | | | | | Global test | 0.003 |

7 degrees of freedom
ns = not significant
Haplotype analysis demonstrated that the h4 haplotype (table 2) was positively associated with risk for PD, and the h1 haplotype is negatively associated with risk.

A Multi-locus genotype PDTsum demonstrates the genotype 22-1,2 is the genotype giving the most significant allele association. (See table 3).

TABLE 3
Multilocus genotype PDTsum analysis

Genotype

| A | B | Z | p-value |
|---|---|---|---|
| 1, 1 | 1, 1 | −2.480 | 0.013 |
| 1, 1 | 1, 2 | 0.000 | 1.000 |
| 1, 2 | 1, 1 | −0.912 | 0.362 |
| 1, 2 | 1, 2 | 0.000 | 0.946 |
| 2, 2 | 1, 1 | 0.697 | 0.486 |
| 2, 2 | 1, 2 | 2.785 | 0.005 |
| 2, 2 | 2, 2 | 0.810 | 0.423 |

A rs1989754
B 8p0215

Linkage disequilbrium (LD) analysis demonstrated that the two associated SNPs are in LD with each other (table 4).

TABLE 4
Linkage disequilibrium test of FGF 20 SNPs
LD test---R2

| | RS1989756 | RS1989754 | RS1721100 | 8p0215 |
|---|---|---|---|---|
| Affected | | | | |
| 8P0217 | 0.086 | 0.652 | 0.045 | 0.097 |
| RS1989756 | | 0.058 | 0.018 | 0.009 |
| RS1989754 | | | 0.268 | 0.073 |
| RS1721100 | | | | 0.259 |
| Unaffected | | | | |
| 8P0217 | 0.081 | 0.677 | 0.069 | 0.09 |
| RS1989756 | | 0.058 | 0.018 | 0.004 |
| RS1989754 | | | 0.267 | 0.058 |
| RS1721100 | | | | 0.245 |

LD test---D prime

| | 8P0217 | RS1989756 | RS1989754 | RS1721100 | 8p0215 |
|---|---|---|---|---|---|
| Affected | | | | | |
| 8P0217 | | 1 | 0.986 | 0.315 | 0.968 |
| RS1989756 | | | 1 | 0.724 | 1 |
| RS1989754 | | | | 0.943 | 0.961 |
| RS1721100 | | | | | 1 |
| Unaffected | | | | | |
| 8P0217 | | 1 | 0.979 | 0.399 | 1 |
| RS1989756 | | | 1 | 0.75 | 0.717 |
| RS1989754 | | | | 0.94 | 0.873 |
| RS1721100 | | | | | 1 |

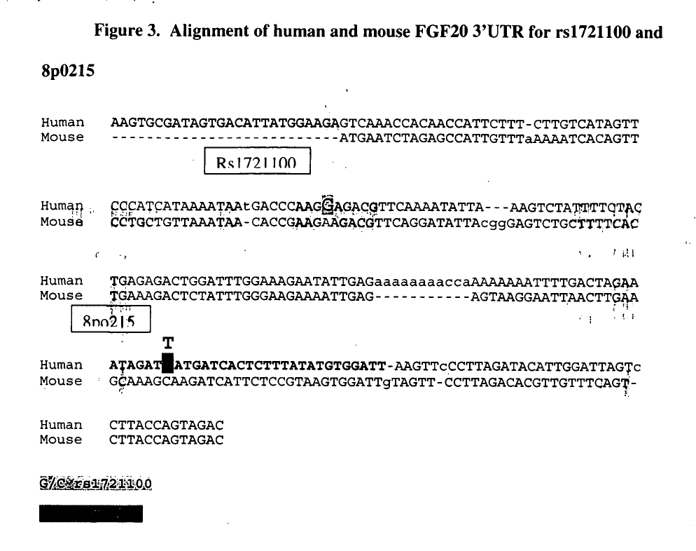

Thus, either or both could illustrate increasing risk for Parkinson's disease, either independently or through interaction between them. The SNP 8p0215 we found lies in a highly conserved region of the FGF20 gene, and lies within a PUF binding site, the SNP highlighted in FIG. 3. PUF are proteins that are involved in mRNA stabilization.

In describing the mutations disclosed herein in the novel nucleic acids described herein, and the nucleotides encoding the same, the naming method is as follows: [nucleic acid replaced] [nucleic acid number in sequence of known sequence][alternate nucleic acid]. For example, for the 817^{th} position is cytosine and is replaced with a thymine.

Materials and Methods

A total of 644 families were genotyped. Of these families, 289 were multiplex families (2 or more affected individuals within a family), and 355 were singleton familes (I affected individual within a family). Exonic, intronic and untranslated regions (UTR) were screened for SNPs by sequencing pools of individuals.

Microarray Gene Expression Study: Total RNA was extracted using TRIzol reagent (Invitrogen, Carlsbad, Calif.) according to the manufacturer's instructions. To label the RNA for hybridization to the microarray chip, 7 μg of total RNA were used for double-stranded cDNA synthesis using the SuperScript Choice System (Gibco BRL Life Technologies, Rockville, Md.) in conjunction with a T7-(dT)-24 primer (Geneset Oligos, La Jolla, Calif.). The cDNA was purified using Phase Lock Gel (3 Prime, Inc., Boulder, Colo.). In vitro transcription was performed to produce biotin-labeled cRNA using a BioArray HighYield RNA Transcript Labeling Kit (Affymetrix, Santa Clara, Calif.) according to the manufacturer's instructions. The biotinylated RNA was cleaned using the RNeasy Mini kit (Qiagen, Valencia, Calif.). See, Lockhart et al., Nat. Biotechnol. 14, 1675 (1996); and Warrington et al., Physiol Genomics 2, 143 (2000).

To probe the microarray, 20 μg of biotinylated cRNA was fragmented and hybridized to microarrays (GeneChip Human Genome U133A array, Affymetrix) using previously described protocols. See, Lockhart et al. The intensity of all features of microarrays was recorded and examined for artifacts (Affymetrix GeneChip® Software v 4.0). O'Dell et al., Eur. J. Hum. Genet 7, 821 (1999). Quantitative gene expression values measured by the average difference between the hybridization intensity with the perfect match probe sets and the mismatch probe sets were then multiplied by a scaling factor to make the mean expression level on the microarray equal to a target intensity of 100. The Affymetrix software to normalize the gene expression levels automatically performs this scaling.

For quality control, all arrays were visually inspected to exclude hybridization artifacts. To control for partial RNA degradation, 3′/5′ end ratios for the housekeeping genes actin and GAPDH were examined. Arrays with high 3′/5′ end ratios suggestive of partial RNA degradation were excluded from further analysis.

Microarray Data Analysis: Since genes with low signal intensity often cause high variability between arrays and Northern blots usually do not confirm positive results for genes with signal intensity less than 500, only genes with average expression intensities of =500 were considered for further analysis. A log₂ (logarithm base 2) was used for data normalization, so data within each chip are in agreement with normal distribution. A two-sample t-test was used to examine whether the gene expression between case and control groups is significantly different. Disease status was randomly assigned to each sample for 1000 times to estimate an empirical p-value for each gene. A nominal significance level of 0.05 was compared with the empirical p-values to declare a result significant.

SNP detection and genotyping: Public domain databases (Japanese JSNP, http://snp.ims.u-tokyo.ac.ip, NCBI dbSNP, http://www.ncbi.nlm.nih.gov/SNP/, and Applied Biosystems http://www.appliedbiosystems.com) were utilized to identify SNPs located in or near the candidate genes. All other SNPs were genotyped using the assays-on-demand from Applied Biosystems (ABI, Foster City, Calif.). Genomic DNA was extracted from whole blood using the PureGene system (Gentra Systems, Minneapolis, Minn.) and genotyped using the TaqMan allelic discrimination assay. See, Saunders et al., Neurol. 43, 1467 (1993); and Vance et al., Approaches to Gene Mapping in Complex Human Diseases, (Wiley-Liss, New York, 1998), chap. 9.

Association Analysis: All SNPs were tested for Hardy-Weinberg equilibrium (HWE) and linkage disequilibrium (LD) in the affected group (one affected from each family) and the unaffected group (one unaffected from each family). An exact test implemented in Genetic Data Analysis (GDA) program was used to test HWE, in which 3,200 replicate samples were simulated for estimating the empirical P value. See, Zaykin et al., Genetica, 96, 169 (1995). The GOLD (Graphical Overview of Linkage Disequilibrium) program was used to estimate the Pearson correlation (r 2) of alleles for each pair of SNPs as the measurement of LD. See, Abecasis et al. The higher the r² (0<r²<1), the stronger the LD. In general, r²>0.3 is considered to be a minimum useful value for detecting association with an unmeasured variant related to disease risk by genotyping a nearby marker in LD with that variant. See, Ardlie et al., Nat. Rev. Genet. 3, 299 (2002). Additionally, the Pedigree Disequilibrium Test (PDT) and GenoPDT were utilized as statistical methods.

The orthogonal model takes information from a general pedigree. It can incorporate covariate effects when necessary. The association between the marker and age-at-onset was identified by testing within family effect, which is equivalent to the additive effect of the marker locus. The empirical p-values were computed through 1000 permutations to avoid false-positive results.

In the specification, there has been disclosed typical preferred embodiments of the invention and, although specific terms are employed, they are used in a generic and descriptive sense only and not for purposes of limitation of the scope of the invention being set forth in the following claims.

1. A method of screening a human subject for risk of Parkinson's disease, comprising:
detecting the presence or absence of a mutation at a position selected from the group consisting of position 817 and at position 5 in rs1989754 in Chromosome 8 collected from a biological sample of said subject; and
determining that the subject is at an increased risk of Parkinson's disease due to the presence of said mutation in Chromosome 8.

2. The method according to claim 1, wherein said mutation at position 817 is C817T.

3. The method according to claim 1, wherein said mutation at position 5 is C5G.

4. A method of screening a subject for Parkinson's disease comprising:
detecting the presence or absence of at least one or more markers linked to Parkinson's disease, wherein the presence of said marker indicates that the subject is afflicted with or at risk of developing Parkinson's disease, and wherein said marker is located in the linkage peak Fibroblast growth factor 20(FGF20).

5. The method according to claim 4, wherein said marker is linked to age of onset of Parkinson's disease.

6. The method according to claim 4, wherein said method is a diagnostic method.

7. The method according to claim 4, wherein said method is a prognostic method.

8. The method according to claim 4, wherein said Parkinson's disease is early-onset Parkinson's disease.

9. The method according to claim 4, wherein said subject is human.

10. A method for diagnosing a subject as having Parkinson's disease, or as having a predisposition to Parkinson's disease comprising:
determining the presence or absence of an allele of a polymorphic marker in the subject, wherein (i) the allele is associated with a phenotypic marker of Parkinson's disease, and wherein (ii) the polymorphic marker is within a segment selected from the group consisting of:
a segment of chromosome 8 bordered by 8p21.3 to 8p22 and D8S373.

11. The method according to claim 10, wherein said determining the presence or absence of an allele of a polymorphic marker in the subject is performed utilizing DNA or RNA.

12. The method according to claim 10, wherein said marker is linked to age of onset of Parkinson's disease.

13. The method according to claim 10, wherein said method is a diagnostic method.

14. The method according to claim 10, wherein said method is a prognostic method.

15. The method according to claim 10, wherein said Parkinson's disease is early-onset Parkinson's disease.

16. The method according to claim 10, wherein said subject is human.

17. An assay for detecting a gene related to an age of onset disorder comprising:
providing a biological sample comprising genomic DNA from a patient suspected of having or at risk for developing said age of onset disorder for Parkinson's disease;
using a probe directed toward to a region of a polymorphic marker in the subject, wherein (i) the marker is associated with a phenotypic marker of Parkinson's disease, and wherein (ii) the polymorphic marker is within a segment selected from the group consisting of:
a segment of chromosome 8 bordered by FGF20; and
detecting duplications in the region of the genomic sequence of the group of chromosomes listed above.

18. The assay of claim 17, where said age of onset disease is Parkinson's disease.

19. The assay of claim 17, wherein said Parkinson's disease is early-onset Parkinson's disease.

20. A nucleic acid molecule encoding a Fibroblast Growth Factor 20 (FGF20) having a mutation that is associated with Parkinson's Disease, wherein said mutation is selected from the group consisting of (i) cytosine at position 5 in rs 1989754 and (ii) thymine at position 817 as designated as 8p0215.

21. An oligonucleotide of from 5 to 50 bases that hybridizes to a nucleic acid of claim 20.


SEQUENCE LISTING (7 sequences)

SEQ ID NO: 1 - DNA, 1016 bp, Homo sapiens
  CDS (134)..(766)
agcgacctcagaggagtaaccgggccttaactttttgcgctcgttttgctataatttttctctatccacctccatcccacccccacaacactctttactgggggggtcttttgtgttccggatctccccctccatggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttgaagtgcgatagtgacattatggaagagtcaaaccacaaccattctttcttgtcatagttcccatcataaaataatgacccaaggagacgttcaaaatattaaagtctattttctactgagagactggatttggaaagaatattgagaaaaaaaaccaaaaaaaattttgactagaaatagatcatgatcactctttatatgtggattaagttcccttagatacattggattagtccttaccagtagac

SEQ ID NO: 2 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 3 - DNA, 358 bp, Homo sapiens
tcctttgacattgctagcaggttaactaatagaatggaaacttcagctatggggaaagatcctgggatattagaaccggagagcaccccatctttgtacagaaaactaagcctcagactgatgaaggcactttctagttacacagctagtgaggaagtcattaacaggagagaccctcccgatctagtatcttaacagacactgccttaacaatcattctcttgtttcttttaaccccttctcttcccaggcactgccggaggtattctgaaacacgtccgtctgtgttcccacccatatcttctttcgctttcccatttcctctttcctaaagtcgataccaagatacttgctttca

SEQ ID NO: 4 - DNA, 237 bp, Homo sapiens
gttgcacaatttccaaagaggagcttggctgaagaactaggcatgctcagtagccgggtggtcttcctcctcccccacccctccccccctttccttttcttttctcacccacatagaacttaggagctgagggaacctcagacaggtgagccctacaggtagcgaatgtgcccacggaaagttaatctgctacctcttcaggtgaacatttgcaagtctctaggtagacacgtaaat

SEQ ID NO: 5 - DNA, 52 bp, Homo sapiens
  variation (26)..(26): SNP rs1989754
agctcctctttggaaattgtgcaacgtgaaagcaagtatcttggtatcgact

SEQ ID NO: 6 - DNA, 248 bp, Homo sapiens
  variation (84)..(84): SNP rs1721100
aagtgcgatagtgacattatggaagagtcaaaccacaaccattctttcttgtcatagttcccatcataaaataatgacccaaggagacgttcaaaatattaaagtctattttctactgagagactggatttggaaagaatattgagaaaaaaaaccaaaaaaaattttgactagaaatagatcatgatcactctttatatgtggattaagttcccttagatacattggattagtccttaccagtagac

SEQ ID NO: 7 - DNA, 213 bp, Mus musculus
atgaatctagagccattgtttaaaaatcacagttcctgctgttaaataacaccgaagaagacgttcaggatattacgggagtctgcttttcactgaaagactctatttgggaagaaaattgagagtaaggaattaacttgaagcaaagcaagatcattctccgtaagtggattgtagttccttagacacgttgtttcagtcttaccagtagac